(19) United States
(12) Patent Application Publication
(10) Pub. No.: US 2010/0099957 A1
(43) Pub. Date: Apr. 22, 2010

(54) PHYSIOLOGICAL PARAMETER MONITORING DEVICE

(76) Inventor: Wei-Kung WANG, Taipei (TW)

Correspondence Address: PATENTTM.US, P. O. BOX 82788, PORTLAND, OR, 97282-0788, US

(21) Appl. No.: 12/574,558

(22) Filed: Oct. 6, 2009

(30) Foreign Application Priority Data
Oct. 20, 2008 (TW) 097140254

(51) Int. Cl. A61B 5/02; A61B 5/1455

(52) U.S. Cl. 600/301; 600/500; 600/324

(57) ABSTRACT

A physiological parameter monitoring device has a physiological parameter detector and an analyzer. The physiological parameter detector has a wrist belt, a pulse sensor mounted to an inner side of the wrist belt to output a pulse sensing signal and an analog to digital converter. The pulse sensor has a soft bag used to press wrist artery and a pressure sensor connected to the soft bag to detect an inner pressure change according to the wrist artery and then output the pulse sensing signal. The analog to digital converter is electronically connected to the pressure sensor to converter the pulse sensing signal to digital signal. The analyzer links to the physiological parameter to determine a health condition according to the digital signal of the pulse sensing signal.

### BACKGROUND OF THE INVENTION

1. Field of the Invention

The present invention relates to a physiological parameter detector, and more particularly to physiological parameter monitoring device that conveniently fits on different wrist of human to detect pulse signal and further determine health condition of human.

2. Description of Related Art

Traditionally the pulse feeling machine uses a small transducer with fixed shape, mostly round to fix on the artery. Both the transducer and the artery are much less than 1 cm in dimension. It is hard to align the hard transducer firmly on the uneven artery to detect the pulses of the arterial pressure.

To overcome the shortcomings, the present invention provides a physiological parameter monitoring device to mitigate or obviate the aforementioned problems.

### SUMMARY OF THE INVENTION

The main objective of the present invention is to provide a physiological parameter monitoring device that conveniently fits on different wrist of human to detect pulse signal and further determine health condition of human.

The physiological parameter monitoring device has a physiological parameter detector and an analyzer. The physiological parameter detector has a wrist belt, a pulse sensor mounted to an inner side of the wrist belt to output a pulse sensing signal and an analog to digital converter. The pulse sensor has a soft bag used to press wrist artery and a pressure sensor connected to the soft bag to detect an inner pressure change according to the wrist artery and then output the pulse sensing signal. The analog to digital converter is electronically connected to the pressure sensor to converter the pulse sensing signal to digital signal. The analyzer links to the physiological parameter to determine a health condition according to the digital signal of the pulse sensing signal.

Other objectives, advantages and novel features of the invention will become more apparent from the following detailed description when taken in conjunction with the accompanying drawings.

### BRIEF DESCRIPTION OF THE DRAWINGS

FIG. 1 is a schematic view of a first embodiment of a physiological parameter monitoring device in accordance with the present invention;

FIG. 2 is a schematic view of a second embodiment of a physiological parameter monitoring device in accordance with the present invention;

FIG. 3 is a partial cross sectional view of a physiological parameter monitoring device in accordance with the present invention;

FIG. 4 is a schematic view of a sweat sensor of the physiological parameter monitoring device in accordance with the present invention;

FIG. 5 is a block diagram view of an analyzer of the physiological parameter monitoring device mounted around the wrist;

FIG. 6 is a schematic view of the physiological parameter monitoring device mounted around the wrist of human; and

FIG. 7 is a flow chart of a monitoring procedure built in the analyzer of the physiological parameter monitoring device in accordance with the present invention.

### DETAILED DESCRIPTION OF THE PREFERRED EMBODIMENT

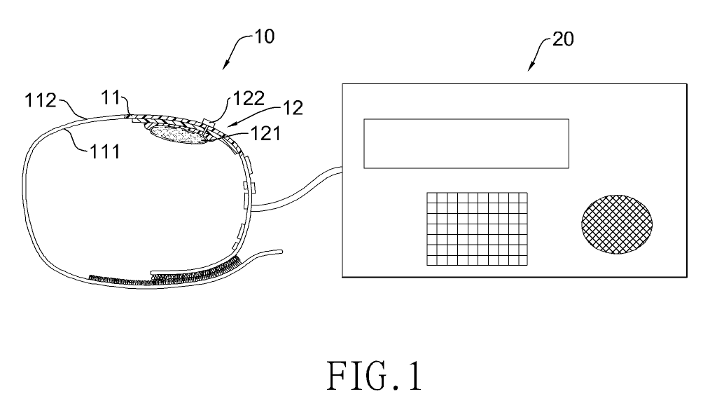

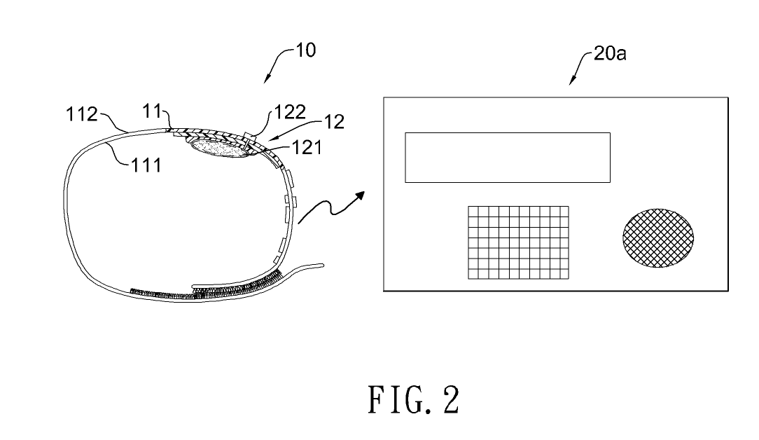

With reference to FIG. 1, a physiological parameter monitoring device in accordance with the present invention has a physiological parameter detector (10) and an analyzer (20) linking to the physiological parameter detector (10). In a first embodiment, the analyzer (20) is connected to the physiological parameter detector (10) through wires (30) or integrated into the physiological parameter detector (10). With reference to FIG. 2, A physiological parameter detector (10a) is wireless connected to the analyzer (20a).

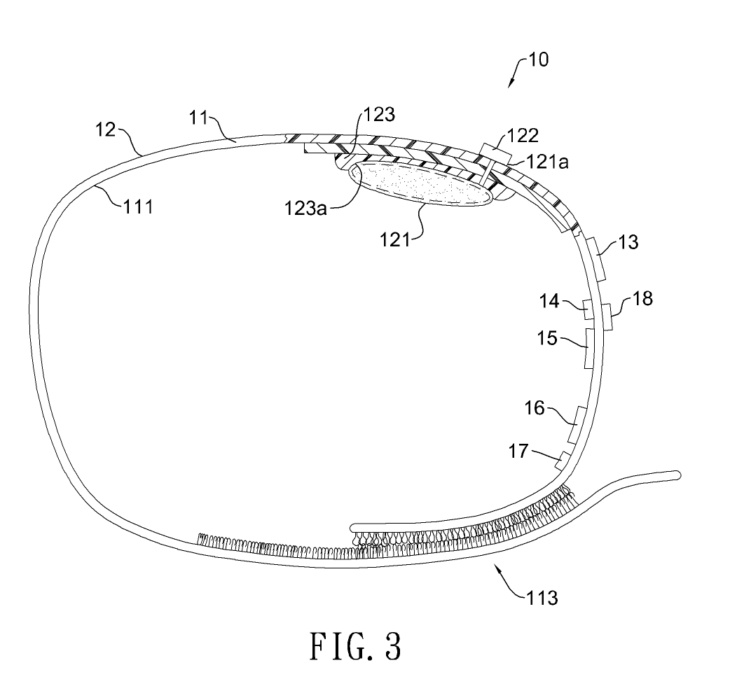

With reference to FIG. 3, the physiological parameter detector (10) has a wrist belt (11), a pulse sensor (12), an electrical conductivity sensor (14), a blood oximetry sensor (15), a temperature sensor(16), an accelerator (17), an alarm unit (18), an analog to digital converter (ADC) (13), and a signal connector (19). The wrist belt (11) has an inner side (111), an outer side (112) and a fixing device (113). A part of the pulse sensor (12), the electrical conductivity sensor (14), the blood oximetry sensor (15), the temperature sensor (16), the accelerator (17), and the alarm device (18) are mounted on the inner side (111) of the wrist belt (11). The ADC (13) and the signal connector (19) are mounted to the outer side (112). The fixing device (113) is an adhesive strap. The wrist belt (11) may be elastic to firmly fit on the wrist. Since the elastic wrist belt (11) has the adhesive strap, the elastic wrist belt (11) suits to different wrists.

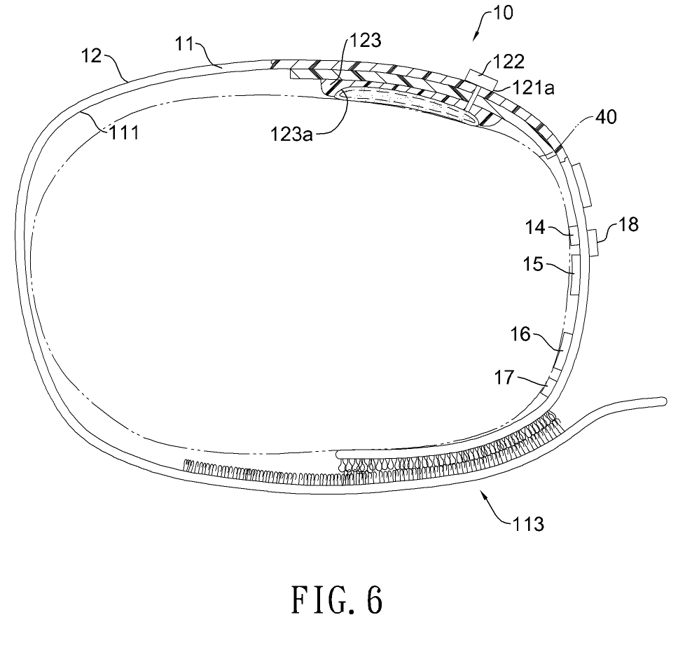

The pulse sensor (12) has a rigid seat (123), a soft bag (121) and a pressure sensor (122). The rigid seat (123) is securely mounted on the inner side (111) of the wrist belt (11) and has a curve face (123a) on which the soft bag (121) mounted. The soft bag (121) further has a hollow tube (121a) extended from one side of the soft bag (121) and communicated with an inside of the soft bag (121) connected to the pressure sensor (122). The hollow tube (121a) passes through the wrist belt (11) and connected to the pressure sensor (122) since the pressure sensor (122) is mounted the outer side (112) of the wrist belt (11). The soft bag (121) may be filled with gas, liquid or free flowing small particles etc. fluid material. With further reference to FIG. 6, when the wrist belt (11) fixes around the wrist (40), the soft bag (121) has to align to the artery (now shown) of the wrist (40) and then the soft bag (121) is tightened to deform to fit the wrist (40). Since the artery is radial, the soft bag (121) may be rectangular or formed of a 2 cm*1 cm elliptical shape. The detecting signal of the pressure sensor (122) is changed according to pulse of the artery to response the pulse signal of the user.

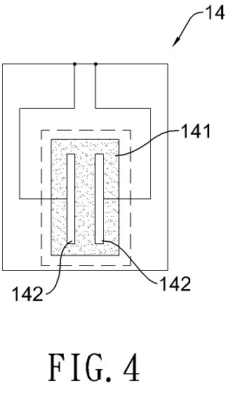

With further reference to FIG. 4, the electrical conductivity sensor (14) is used to detect sweat condition and has a thin water absorption pad (141) and two electrodes (142) mounted to the thin water absorption pad (141). The thin water absorption pad (141) absorbs sweat from the wrist and then resistance between the two electrodes (142) is reduced. Therefore, the resistance change between the two electrodes (142) responses a present sweat condition of the user. Since salinity of the diabetic's sweat is lower than that of the health people's sweat, the thin water absorption pad (141) contains salt powder to increase conductivity of the electrodes.

The blood oximetry sensor (15) has multiple light sources and photo-detectors. The light source may be an LED with a wave length around 660 nm, 800 nm and 940 nm. The photo-detector may be a surface mounted device.

The alarm unit (18) may have a lighting device, a speaker and/or an electrical shocking device.

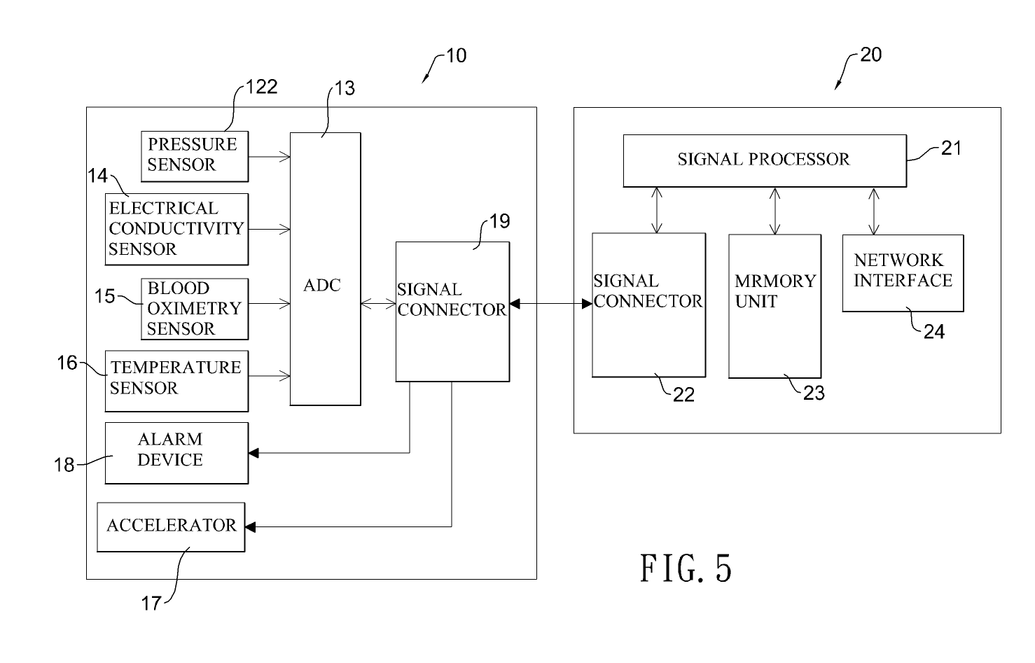

With further reference to FIG. 5, the signal connector is electronically connected to the ADC (13), and the ADC (13) is electronically connected to the pressure sensor (122) of the pulse sensor (12), electrical conductivity sensor (14), the blood oximetry sensor (15), the temperature sensor (16) and the accelerator (17). The alarm unit (18) is electronically connected to the signal connector (19). Since the pressure sensor (122), electrical conductivity sensor (14), the blood oximetry sensor (15), the temperature sensor (16) and the accelerator (17) respectively output an analog sensing signals, the ADC (13) converters the analog sensing signals to corresponding digital signals. The ADC (13) outputs the digital signals to the signal connector (19).

The analyzer (20) has a signal processor (21), a signal connector (22), a memory unit (23) and a network interface (24). The signal connector (22) of the analyzer (20) is connected to the signal connector (19) of the physiologic parameter detector (10). Each of the signal connectors (19, 22) of the physiological parameter detector (10) and the analyzer (20) may be transceiver, such as blue tooth device, so as to wireless link to each other. The processor (21) of the analyzer (20) is electronically connected to the signal connector (22), the memory unit (23), the network interface (24). A monitoring procedure is built in the processor (21). The processor (21) obtains digital sensing signals from the physiological parameter detector (10) and also links to Internet through the network interface (24). Therefore, the processor (10) analyzes heat rate, blood oxygen, body temperature, sweat condition and so on and stores these parameters in the memory unit. The parameters are re-stored after the processor (21) received a next set of the parameters. Further, the processor (21) further presets various standard physiological signals according to different gender, age, body positions. The processor (21) also uses heart beat as one period to analyze the digital signal corresponding to the sensing signal from the pressure sensor (122). The processor (21) transforms the digital signal corresponding sensing signal of the processor sensor (122) to harmonics of the heart beat by the Fourier transfer equation. Amplitude and phase of the transformation is compared with the corresponding standard signals established by grouping subjects into man or woman, age, measuring from the artery as target at different part of the body.

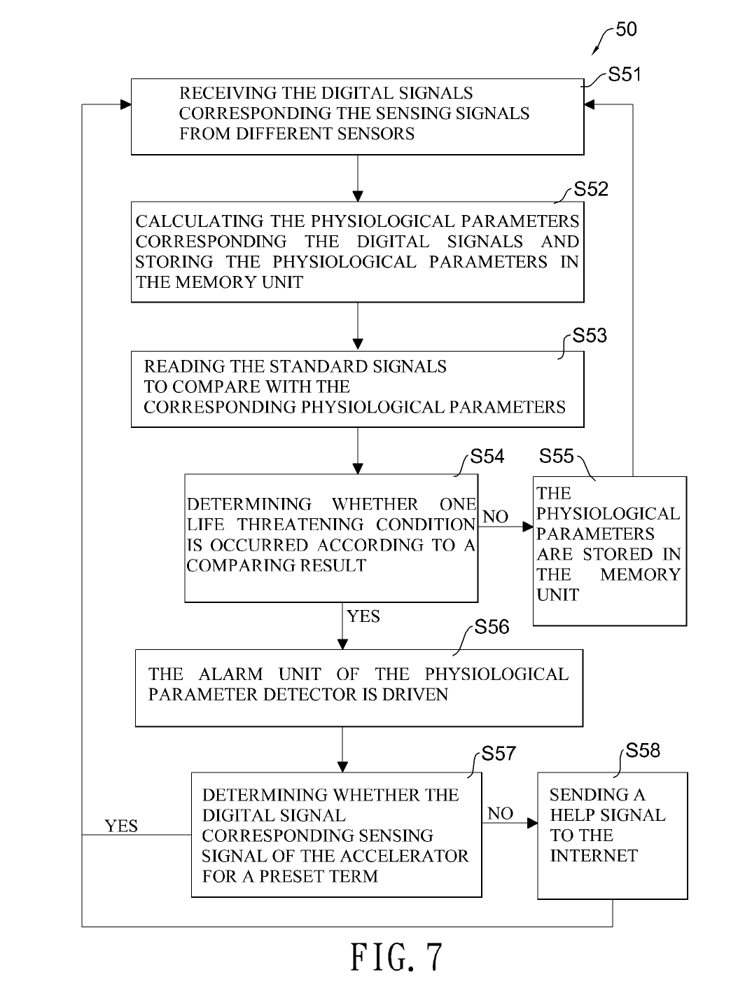

Person with hyperglycemia symptoms will experience any of the following nervousness, sweating, trembling, palpitation, lowering body temperature and etc. For more severe conditions, person may lose conscious and become life threatening danger, this device can be worn like a watch during sleep. The present invention monitors theses symptoms easily and immediately. That is, the nervousness and trembling can be detected by the accelerator or the pressure sensor, the palpitation can be detected by the pressure sensor as well as the blood oximetry, and the sweating can be detected by the electrical conductivity sensor. While the lowering body temperature can be detected by the temperature sensor. The more dangerous life threatening cases can be detected by the weakening or losing of pulse, the sudden drop of the blood oxygen (SPO₂) and abnormal changing body temperature. With reference to FIGS. 5, 6 and 7, the processor (21) cyclically executes the monitoring procedure (50) having following steps.

When the different digital signals corresponding the digital signals corresponding the sensing signals from different sensors are received by the processor (21) (S51), the corresponding physiological parameters are calculated (S52). The processor (21) then reads the proper standard signals for the current user (S53) and compares with the physiological parameters to determine whether the user has life threatening condition (S54). If there is no life threatening condition, the physiological parameters are stored in the memory unit (S55) and the processor (21) re-executes the monitoring procedure from the step of receiving digital signals (S51). If not, the user may lose conscious and the processor (21) have to drive the alarm unit (18) of the physiological parameter detector (10) (S56). Since the alarm unit (18) has the lighting device, the speaker and the electric shocking unit, the alarm unit (18) first drives the lighting device and the speaker to light and output audio signal to wake up the user. Then, the processor (21) waits to receive the digital signal corresponding sensing signal of the accelerator (17) for a preset term (S57) to detect the movement of the user. If the processor (21) received the digital signal corresponding sensing signal of the accelerator (17) during the preset term, the user is wake up and the processor (21) re-executes the monitoring procedure from the step of receiving digital signals (S51). On the contrary, when the specific term is ended and digital signal corresponding sensing signal of the accelerator (21) is not received, the processor (21) outputs a help signal to Internet through the network interface (24) (S58). The processor (21) re-executes the monitoring procedure from the step of receiving digital signals (51).

Based on the foregoing description, the wrist belt is tightened around the user's wrist so the soft bag is securely pressed on a location of the artery of the wrist to accurately monitor the pulse signal and other physiological signals. When the user stays alone or is sleeping, the physiological parameter monitoring device in accordance with the present invention can immediately monitors the user's health condition and automatically wake up the user when any life threatening condition is determined. In addition, the physiological parameter monitoring device further sends the help signal to the Internet to call help when the physiological parameter monitoring device determines that the user does not wake up. Therefore, the physiological parameter monitoring device is easily to use and monitor the use's life and health condition all the time.

Even though numerous characteristics and advantages of the present invention have been set forth in the foregoing description, together with details of the structure and function of the invention, the disclosure is illustrative only. Changes may be made in detail, especially in matters of shape, size, and arrangement of parts within the principles of the invention to the full extent indicated by the broad general meaning of the terms in which the appended claims are expressed.

What is claimed is:

1. A physiologic parameter monitoring device, comprising:
a physiological parameter detector having:
a wrist belt having an inner side, an outer side and a fixing device;
a pulse sensor mounted to the inner side of the wrist belt to output a pulse sensing signal, and having a soft bag adapted to press wrist artery and a pressure sensor connected to the soft bag to detect an inner pressure change according to the wrist artery and then output the pulse sensing signal; and
an analog to digital converter electronically connected to the pressure sensor of the pulse sensor to converter the pulse sensing signal to digital signal; and
an analyzer linking to the physiological parameter to determine a health condition according to the digital signal of the pulse sensing signal.

2. The physiologic parameter monitoring device as claimed in claim 1, wherein the pulse sensor further comprises a rigid seat securely mounted on the inner side of the wrist belt and has a curve face on which the soft bag mounted.

3. The physiologic parameter monitoring device as claimed in claim 2, wherein the soft bag further has a hollow tube extended from one side of the soft bag and communicates with an inside of the soft bag, wherein the inside of the soft bag is filled with fluid material.

4. The physiologic parameter monitoring device as claimed in claim 3, wherein the fluid material is gas, liquid or free flowing small particles.

5. The physiologic parameter monitoring device as claimed in claim 1, wherein the fixing device is an adhesive strap and the wrist belt is elastic adapted to firmly fit on the wrist.

6. The physiologic parameter monitoring device as claimed in claim 4, wherein the fixing device is an adhesive strap and the wrist belt is elastic adapted to firmly fit on the wrist.

7. The physiologic parameter monitoring device as claimed in claim 1, wherein the physiologic parameter detector further comprises:
an electrical conductivity sensor, a blood oximetry sensor, a temperature and an accelerator respectively mounted on the inner side of the belt and electronically connected to the analog to digital converter to output sensing signals to the analog to digital converter, wherein the analog to digital converter converts the sensing signals to corresponding digital signals;
a signal connector electronically connected to the analog to digital converter to connect the analyzer; and
an alarm unit electronically connected to the signal connector.

8. The physiologic parameter monitoring device as claimed in claim 7, wherein
the blood oximetry sensor has multiple light sources and photo-detectors, wherein each light source is an LED with a wave length selected from one of 660 nm, 800 nm and 940 nm, and the photo-detector is a surface mounted device;
the electrical conductivity sensor has a thin water absorption pad and two electrodes mounted to the thin water absorption pad;
the alarm unit may have a lighting device, a speaker or an electrical shocking device.

9. The physiologic parameter monitoring device as claimed in claim 8, wherein the soft bag may be rectangular or formed of a 2 cm*1 cm elliptical shape.

10. The physiologic parameter monitoring device as claimed in claim 8, wherein the thin water absorption pad further contains salt powder to increase conductivity of the electrodes.

11. The physiologic parameter monitoring device as claimed in claim 7, wherein the analyzer comprises:
a signal connector connected to the signal connector of the physiologic detecting device;
an internet interface linking to the Internet;
a processor electronically connected to the signal connector of the analyzer to receive the digital signals corresponding sensing signals and calculate physiological parameters corresponding digital signals, and electronically connected to the internet interface to link to the Internet, and has a monitoring procedure; and
a memory unit electronically connected to the processor to store the physiological parameters.

12. The physiologic parameter monitoring device as claimed in claim 11, wherein each of the signal connectors of the physiological parameter detecting device and the analyzer is a transceiver.

13. The physiologic parameter monitoring device as claimed in claim 12, wherein the transceiver is a blue tooth device.

14. The physiologic parameter monitoring device as claimed in claim 11, wherein the processor further presets various standard physiological signals according to different gender, age, body positions, and uses heart beat as one period to analyze the digital signal corresponding to the sensing signal from the pressure sensor, and transforms the digital signal corresponding sensing signal of the processor to harmonics of the heart beat by the Fourier transfer equation.

15. The physiologic parameter monitoring device as claimed in claim 14, wherein the processor cyclically executes the monitoring procedure having steps of:
receiving the digital signals corresponding the sensing signals from different sensors;
calculating the physiological parameters corresponding the digital signals and storing the physiological parameters in the memory unit;
reading the standard signals to compare with the corresponding physiological parameters;
determining whether one life threatening condition is occurred according to a comparing result, wherein if a determining result is negative, the physiological parameters are stored in the memory unit and then go back to the step of receiving the digital signals; on the contrary, the alarm unit of the physiological parameter detector is driven;
determining whether the digital signal corresponding sensing signal of the accelerator for a preset term, wherein if a determining result is positive and then go back to the step of receiving different digital signals; one the contrary, a help signal is sent to the Internet; and
returning to the step of receiving different digital signals.

16. The physiologic parameter monitoring device as claimed in claim 1, wherein the analyzer is integrated to the physiological parameter detector.

17. The physiologic parameter monitoring device as claimed in claim 15, wherein the analyzer is integrated to the physiological parameter detector.
